(19) United States
(12) Patent Application Publication
(10) Pub. No.: US 2026/0106040 A1
(43) Pub. Date: Apr. 16, 2026

(54) MODEL GENERATION DEVICE, MODEL GENERATION METHOD, AND NON-TRANSITORY COMPUTER-READABLE MEDIUM

(71) Applicant: NEC Corporation, Tokyo (JP)

(72) Inventors: Keisuke SUZUKI, Tokyo (JP); Yuki KOSAKA, Tokyo (JP)

(73) Assignee: NEC Corporation, Tokyo (JP)

(21) Appl. No.: 19/347,015

(22) Filed: Oct. 1, 2025

(30) Foreign Application Priority Data
Oct. 11, 2024 (JP) 2024-178734

(51) Int. Cl. G16H 50/30; G06F 30/27; G16H 50/70

(52) U.S. Cl. CPC G16H 50/30; G06F 30/27; G16H 50/70

(57) ABSTRACT

As a data set of a health condition data value which is a value related to a health condition, two data sets having a temporal relationship are used, a start point is taken from one data set of the two data sets, an end point is taken from the other data set, and an average of a Gaussian distribution followed by the health condition data value during a temporal change from the start point to the end point is set to a continuous function for weighting the health condition data value such that a weight on the health condition data value increases as a data density estimated by kernel density estimation increases, and learning of a model is performed by conditional flow matching. In addition, by estimating health conditions using the learned model, decision making related to health by predicted targets can be supported.

### INCORPORATION BY REFERENCE

This application is based upon and claims the benefit of priority from Japanese patent application No. 2024-178734, filed on Oct. 11, 2024, the disclosure of which is incorporated herein in its entirety by reference.

### TECHNICAL FIELD

The present disclosure relates to a model generation device, a model generation method, and a non-transitory computer-readable medium.

### BACKGROUND ART

It is conceivable that time series data of health conditions of individuals are aggregated and used for training a model for estimating the health condition such as predicting the health condition.

For example, JP 2014-178800 A discloses a method of predicting a future health rank of a prediction target using a hidden Markov model by age group and gender obtained by training time series data of medical examination results for six years for 5000 individuals for each age group and gender. In the method disclosed in JP 2014-178800 A, each hidden Markov model has six conditions, and the six conditions are classified into three health ranks of “health”, “caution needed”, and “required examination (onset)”. Then, in the method disclosed in JP 2014-178800 A, the health rank of the prediction target is predicted using the hidden Markov model selected according to the age and gender of the prediction target and the time series data belonging to the population of the same age and gender as the prediction target.

### SUMMARY

It is considered that it is difficult to collect the time series data of the health condition of an individual, for example, because it takes time to collect data, and there are reasons such as privacy. It is conceivable that data (that is, data that is not time series data) indicating the condition of an individual belonging to a specific group at one time point, such as measurement data in one medical examination of an individual of a specific age, is easier to collect than time series data of the health condition of an individual.

The model for estimating the health condition can be trained using the data indicating the condition of the individual belonging to the specific group at one time point. At that time, if the characteristic considered to be the characteristic related to the temporal change of the value related to the health condition can be reflected in training the model, it is expected that the model can estimate the health condition with higher accuracy.

An example object of the present disclosure is to provide a model generation device, a model generation method, and a program capable of solving the above-described problems.

According to a first example aspect of the present disclosure, a model generation device includes at least one memory storing instructions, and at least one processor configured to execute the instructions to acquire a first data set that is a data set of first health condition data values that are health condition data values for each individual belonging to a first group among health condition data values that are values related to health condition, and a second data set that is a data set of second health condition data values that are health condition data values for each individual belonging to a second group that is a group having a temporal relationship with the first group, set a path of temporal change of an average of a Gaussian distribution set as a probability distribution followed by a health condition data value during temporal change from the first health condition data value to the second health condition data value for each combination of the first health condition data values and the second health condition data values, to a continuous function that connects the first health condition data value and the second health condition data value and performs weighting on the health condition data value in such a way that a weight on the health condition data value increases as a data density estimated by kernel density estimation increases, and set a flow indicating a direction vector of temporal change of the health condition data value with the first health condition data value as a start point and the second health condition data value as an end point in such a way that the health condition data value follows a Gaussian distribution whose average is indicated by the continuous function, and perform optimization calculation of a parameter value of a model for calculating a direction vector of a temporal change of the health condition data value in such a way that a direction vector calculated using the model approaches a direction vector indicated by the flow for all the combinations of the first health condition data values and the second health condition data values.

According to a second example aspect of the present disclosure, a model generation method causes a computer to perform acquiring a first data set that is a data set of first health condition data values that are health condition data values for each individual belonging to a first group among health condition data values that are values related to health condition, and a second data set that is a data set of second health condition data values that are health condition data values for each individual belonging to a second group that is a group having a temporal relationship with the first group, setting a path of temporal change of an average of a Gaussian distribution set as a probability distribution followed by a health condition data value during temporal change from the first health condition data value to the second health condition data value for each combination of the first health condition data values and the second health condition data values, to a continuous function that connects the first health condition data value and the second health condition data value and performs weighting on the health condition data value in such a way that a weight on the health condition data value increases as a data density estimated by kernel density estimation increases, and setting a flow indicating a direction vector of temporal change of the health condition data value with the first health condition data value as a start point and the second health condition data value as an end point in such a way that the health condition data value follows a Gaussian distribution whose average is indicated by the continuous function, and performing optimization calculation of a parameter value of a model for calculating a direction vector of a temporal change of the health condition data value in such a way that a direction vector calculated using the model approaches a direction vector indicated by the flow for all the combinations of the first health condition data values and the second health condition data values.

According to a third example aspect of the present disclosure, a non-transitory computer-readable medium stores a program that causes a computer to execute acquiring a first data set that is a data set of first health condition data values that are health condition data values for each individual belonging to a first group among health condition data values that are values related to health condition, and a second data set that is a data set of second health condition data values that are health condition data values for each individual belonging to a second group that is a group having a temporal relationship with the first group, setting a path of temporal change of an average of a Gaussian distribution set as a probability distribution followed by a health condition data value during temporal change from the first health condition data value to the second health condition data value for each combination of the first health condition data values and the second health condition data values, to a continuous function that connects the first health condition data value and the second health condition data value and performs weighting on the health condition data value in such a way that a weight on the health condition data value increases as a data density estimated by kernel density estimation increases, and setting a flow indicating a direction vector of temporal change of the health condition data value with the first health condition data value as a start point and the second health condition data value as an end point in such a way that the health condition data value follows a Gaussian distribution whose average is indicated by the continuous function, and performing optimization calculation of a parameter value of a model for calculating a direction vector of a temporal change of the health condition data value in such a way that a direction vector calculated using the model approaches a direction vector indicated by the flow for all the combinations of the first health condition data values and the second health condition data values.

An example advantage according to one aspect of the present disclosure is that it is possible to train a model for estimating a health condition by using data indicating the health condition at one time point of each of individuals belonging to a specific group and reflecting a characteristic considered to be a characteristic related to a temporal change of a value related to the health condition in training the model.

### BRIEF DESCRIPTION OF DRAWINGS

The above and other aspects, features, and advantages of the present disclosure will become more apparent from the following description of certain example embodiments when taken in conjunction with the accompanying drawings, in which:

FIG. 1 is a diagram illustrating an example of a configuration of a model generation device according to at least one example embodiment;

FIG. 2 is a diagram illustrating an example of input and output of data in the model generation device according to at least one example embodiment;

FIG. 3 is a diagram illustrating an example of a procedure of processing performed by the model generation device according to at least one example embodiment;

FIG. 4 is a diagram illustrating an example of a configuration of a model generation device according to at least one example embodiment;

FIG. 5 is a diagram illustrating an example of a procedure of processing in the model generation method according to at least one example embodiment; and

FIG. 6 is a diagram illustrating an example of a configuration of a computer according to at least one example embodiment.

### EXAMPLE EMBODIMENT

Hereinafter, example embodiments will be described with reference to the drawings.

### First Example Embodiment

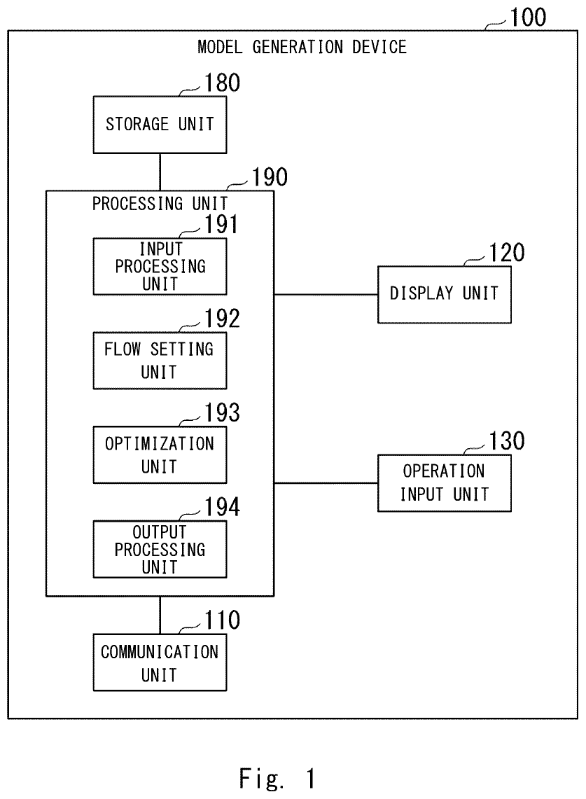

FIG. 1 is a diagram illustrating an example of a configuration of a model generation device according to at least one example embodiment.

In the configuration illustrated in FIG. 1, a model generation device 100 includes a communication unit 110, a display unit 120, an operation input unit 130, a storage unit 180, and a processing unit 190. The processing unit 190 includes an input processing unit 191, a flow setting unit 192, an optimization unit 193, and an output processing unit 194.

The model generation device 100 generates a model for estimating the health condition. For example, with increasing interest in healthcare, the model generation device 100 can be used to predict health conditions.

Using a first data set and a second data set, the model generation device 100 trains a model for estimating a temporal change of a value related to a health condition. The first data set is a data set of first health condition data values that are values related to the health condition for each individual belonging to the first group. The second data set is a data set of second health condition data values that are values related to the health condition for each individual belonging to the second group that is a group having a temporal relationship with the first group. The data included in the first data set is also referred to as first health condition data. The data included in the second data set is also referred to as second health condition data.

The data on the health condition is also referred to as health condition data. The value related to the health condition (the value of the data related to the health condition) is also referred to as a health condition data value. A model for estimating a temporal change in a value related to a health condition is also referred to as a state estimation model.

The health condition data may be vector data indicating values of a plurality of items related to the health condition.

By predicting health conditions using the model generated by the model generation device 100, decision making related to health by predicted targets can be supported.

The health condition here is a physical or mental condition.

Here, the fact that the first group and the second group are in a temporal relationship means that the health condition data value is a value associated with time, and different times are associated between the first health condition data value and the second health condition data value. For example, the first group and the second group may be groups of individuals of different ages.

The second group may be associated with the future time rather than the first group. In this case, the model generation device 100 trains the state estimation model in such a way that the state estimation model predicts a health condition data value that is further in the future than a health condition data value to be input.

For example, in a case where the first group is a group of individuals in their twenties and the second group is a group of individuals in their thirties, the model generation device 100 trains the state transition model in such a way that the state estimation model receives the health condition data value of individuals in their twenties and estimates a temporal change in the health condition data value until the individuals reach their thirties.

Alternatively, the second group may be associated with the past time rather than the first group. In this case, the model generation device 100 trains the state estimation model in such a way that the state estimation model estimates a past health condition data value rather than a health condition data value to be input.

For example, in a case where the first group is a group of individuals in their thirties and the second group is a group of individuals in their twenties, the model generation device 100 trains the state transition model in such a way that the state estimation model receives the health condition data value of the individuals in their thirties and estimates a temporal change in the health condition data value going back to their twenties.

The accuracy of the trained model may be improved by relatively shortening a time width of each group.

For example, a case where a data set of health condition data of an individual of 20 years old or more and less than 30 years old is used as the first data set, and a data set of health condition data of an individual of 30 years old or more and less than 40 years old is used as the second data set is set as a first pattern. A case where a data set of health condition data of individuals of 20 years old or more and less than 25 years old is used as the first data set, and a data set of health condition data of individuals of 30 years old or more and less than 35 years old is used as the second data set is set as a second pattern. The second pattern has less variation in time in the data set than the first pattern, whereby it is expected that the trained model can estimate the temporal change of the health condition data value with relatively high accuracy.

The health condition data used for training the state estimation model by the model generation device 100 does not need to be time series data. The individual belonging to the first group and the individual belonging to the second group may be different individuals.

It is considered that it is difficult to collect the time series data related to the health condition of an individual, for example, because it takes time to collect the data, privacy, and the like. It is conceivable that data (that is, data that is not time series data) indicating the condition of each individual belonging to a specific group at one time point is easier to collect than time series data of the health condition of an individual.

According to the model generation device 100, in this respect, it is expected that data used for training the state estimation model is relatively easily obtained.

The data set of the time series data can also be referred to as a longitudinal data set. The data set associated with the same time can also be referred to as a cross-sectional data set.

The training of the model here is to adjust a parameter value of the model. Learning of the model may also be referred to as training of the model. The training of the model can also be regarded as optimizing a parameter value of the model or optimizing the model. The training of the model can also be regarded as generating a trained model.

The training may be machine learning as an example.

The model generation device 100 may be configured using a computer.

The communication unit 110 communicates with other devices. For example, the communication unit 110 may anonymize the data of the measured value in the periodic medical examination, perform communication with a database device accumulated for each age group of the examinee, and receive the data set for each age group.

For example, the display unit 120 includes a display screen such as a liquid crystal panel or a light emitting diode (LED) panel, and displays various images. For example, the display unit 120 may display information related to training of the state estimation model by the model generation device 100, such as displaying the degree of progress of training the state estimation model.

The operation input unit 130 includes an input device such as a keyboard and a mouse, and receives a user operation. For example, the operation input unit 130 may receive a user operation for performing setting related to training of the state estimation model by the model generation device 100, such as a user operation for setting a variance of a Gaussian distribution assumed as a distribution of the health condition data values.

The storage unit 180 stores various data. For example, the storage unit 180 may store the state estimation model to be trained. The storage unit 180 may store various data acquired by the model generation device 100, which are used for training the state estimation model.

The storage unit 180 is configured using a storage device included in the model generation device 100.

The processing unit 190 controls each unit of the model generation device 100 to perform various types of processing. The function of the processing unit 190 is executed, for example, in a case where a central processing unit (CPU) included in the model generation device 100 reads a program from the storage unit 180 and executes the program.

The input processing unit 191 acquires various data used for training the state estimation model. For example, the input processing unit 191 may acquire a data set of health condition data from another device via the communication unit 110. The input processing unit 191 may acquire, via the operation input unit 130, a parameter value specified by the user related to training of the state estimation model, such as variance of a Gaussian distribution assumed as a distribution of values related to the health condition.

The input processing unit 191 is associated with an example of input processing means.

The flow setting unit 192 performs various settings for training the state estimation model. The flow setting unit 192 is associated with an example of a flow processing means.

The optimization unit 193 trains the state estimation model using the setting by the flow setting unit 192. The optimization unit 193 is associated with an example of optimization means.

The output processing unit 194 outputs the state estimation model trained by the optimization unit 193.

A combination of the flow setting unit 192 and the optimization unit 193 performs training of the parameter value of the state estimation model using a technique of a conditional flow matching (CFM).

Conditional flow matching is a method for optimizing model parameter values in continuous normalizing flow (CNF), which models the change in distribution by differential equations.

Here, for flow u: [0,1]×R^{d}→R^{d}, a solution of a differential equation of Expression (1) with an initial value x∈R^{d} is expressed as φt(x).

$$\begin{matrix}[ Math . 1 ] & \\ \frac{d}{dt} {ϕ}_{t} = {u}_{t} ( {ϕ}_{t} ) & ( 1 )\end{matrix}$$

R represents a real number space. d is an integer of d≥1 and R^{d} represents a d-dimensional real space.

t is a variable having a value of 0≤t≤1. In the model generation device 100, t is treated as a variable indicating the progress of time. t is also referred to as time t.

In the continuous normalizing flow, if probability density functions p₀ and p₁ on R^{d} are given, a model vₜ(x; θ) of the flow uₜ is trained such that φ₁(X)˜p₁ holds for X˜p₀. θ represents a parameter of the model vₜ.

The trained model vₜ approximately calculates (d/dt) φₜ upon receiving x∈R^{d}. That is, the trained model vₜ approximately calculates differential coefficient values indicating paths from x₀ to p₀ to x₁ to p₁. In the model generation device 100, the differential coefficient value calculated by the model vₜ can be regarded as a direction vector of the temporal change of the health condition data value.

In the model generation device 100, the model vₜ(x; θ) is associated with the state estimation model. In the model generation device 100, the point x indicates a health condition data value. The health condition data is indicated by a d-dimensional real number vector.

In the conditional flow matching, optimization calculation of the value of the parameter θ is performed so as to minimize the value of the loss function L_{CFM}(θ) expressed by Expression (2).

$$\begin{matrix}[ Math . 2 ] & \\ {L}_{CFM} ( θ ) = {\int }_{0}^{1} {E}_{x ∼ {p}_{t} ( \cdot {x}_{0} , {x}_{1} ) , ( {x}_{0} , {x}_{1} ) ∼ q} [ {‖ {u}_{t} ( x \vert {x}_{0} , {x}_{1} ) - {v}_{t} ( x ; θ ) ‖}^{2} ] dt & ( 2 )\end{matrix}$$

In the conditional flow matching, the flow uₜ(x|x₀, x₁) in a case where the start point x₀ and the end point x₁ are fixed is considered, and minimization of a squared error ∥uₜ(x|x₀, x₁)−vₜ(x; θ)∥² between the flow uₜ(x|x₀, x₁) and the model vₜ(x; θ) is considered. ∥·∥ represents a norm.

The flow ut(x|x₀, x₁) in a case where the start point x₀ and the end point x₁ are fixed is also referred to as a flow with conditions or a conditional flow.

E represents an expected value.

(x₀, x₁)˜q indicates that the combination of the start point x₀ and the end point x₁ follows the probability distribution q. Here, the probability distribution q is a probability distribution by a combination of the probability distributions p₀ and p₁. That is, it is assumed that the probability distribution q is a distribution on the direct product space R^{d}×R^{d}, the peripheral distribution of the first component (R^{d}) of the direct product space is p₀, and the peripheral distribution of the second component is p₁.

pₜ(·|x₀, x₁) indicates a distribution of the value φₜ at the time t of φ determined by Expression (1) in a case where the initial value is x₀ and the flow is uₜ(·|x₀, x₁) with the pair of (x₀, x₁) being fixed.

x˜pₜ(·|x₀, x₁) indicates that the value of x is given by a random number according to the probability distribution pₜ(·|x₀, x₁).

Here, it is assumed that the probability distribution pₜ(·|x₀, x₁) is a Gaussian distribution at 0≤t≤1. This can be expressed as Expression (3).

$$\begin{matrix}[ Math . 3 ] & \\ {p}_{t} ( x \vert {x}_{0} , {x}_{1} ) = N ( x \vert {μ}_{t} ( {x}_{0} , {x}_{1} ) , {{σ}_{t} ( {x}_{0} , {x}_{1} )}^{2} I ) & ( 3 )\end{matrix}$$

As described above, if the point x follows the Gaussian distribution at 0≤t≤1, it is known that the conditional flow uₜ is determined as Expression (4).

$$\begin{matrix}[ Math . 4 ] & \\ {u}_{t} ( x \vert {x}_{0} , {x}_{1} ) = \frac{{σ}_{t}^{′} ( {x}_{0} , {x}_{1} )}{{σ}_{t} ( {x}_{0} , {x}_{1} )} ( x - {μ}_{t} ( {x}_{0} , {x}_{1} ) ) + {μ}_{t}^{′} ( {x}_{0} , {x}_{1} ) & ( 4 )\end{matrix}$$

σₜ(x₀, x₁) represents the standard deviation of each component (pₜ(·|x₀, x₁) per time t) of pₜ(·|x₀, x₁).

σ′ₜ(x₀, x₁) represents the derivative of σₜ(x₀, x₁) at t.

μₜ(x₀, x₁) represents the average of each component of pₜ(·|x₀, x₁).

μ′ₜ(x₀, x₁) denotes the derivative of μₜ(x₀, x₁) at t.

For example, it is conceivable to set the average μₜ(x₀, x₁) as in Expression (5).

$$\begin{matrix}[ Math . 5 ] & \\ {μ}_{t} ( {x}_{0} , {x}_{1} ) = t {x}_{1} + ( 1 - t ) {x}_{0} & ( 5 )\end{matrix}$$

In a case where the average μₜ(x₀, x₁) is set as in Expression (5), the probability distribution pₜ(x|x₀, x₁) followed by the point x is expressed as in Expression (6).

$$\begin{matrix}[ Math . 6 ] & \\ {p}_{t} ( x \vert {x}_{0} , {x}_{1} ) = N ( x \vert t {x}_{1} + ( 1 - t ) {x}_{0} , {{σ}_{t} ( {x}_{0} , {x}_{1} )}^{2} I ) & ( 6 )\end{matrix}$$

Here, if the characteristic of the distribution of the values related to the human health condition (health condition data values) can be reflected in the probability distribution pₜ(x|x₀, x₁) followed by the point x, it is expected that the state estimation model generated by the model generation device 100 can estimate the health condition with higher accuracy.

It is considered that there is a standard value or a standard range of values related to human health condition, and there is a characteristic that data tends to be concentrated near the standard value or near the standard range. From this, it is conceivable that there is a characteristic that the value related to the human health condition is likely to change through a region having a high data density.

The flow setting unit 192 may set the average μₜ(x₀, x₁) as in Expression (7) in order to reflect that the value related to the human health condition is likely to change through the region with high data density in the conditional flow matching.

$$\begin{matrix}[ Math . 7 ] & \\ {μ}_{t} ( {x}_{0} , {x}_{1} ) = ( 1 - t ) \times \frac{{E}_{x ∼ {p}_{0}} [ x {K}_{t ‖ {x}_{0} - {x}_{1} ‖} ( x - {x}_{0} ) ]}{{E}_{x ∼ {p}_{0}} [ {K}_{t ‖ {x}_{0} - {x}_{1} ‖} ( x - {x}_{0} ) ]} + t \times \frac{{E}_{x ∼ {p}_{1}} [ x {K}_{( 1 - t ) ‖ {x}_{0} - {x}_{1} ‖} ( x - {x}_{1} ) ]}{{E}_{x ∼ {p}_{1}} [ {K}_{( 1 - t ) ‖ {x}_{0} - {x}_{1} ‖} ( x - {x}_{1} ) ]} & ( 7 )\end{matrix}$$

Here, taking the expected value can be regarded as taking a sample average of the values of the expression in [ ] for x according to the probability distribution indicated by the subscript of E.

K represents a kernel function.

Subscripts “t∥x₀-x₁∥” and “(1−t)|x₀-x₁∥” of K both indicate a bandwidth in kernel density estimation.

The bandwidth h can be expressed as Expression (8).

$$\begin{matrix}[ Math . 8 ] & \\ {K}_{h} ( x ) = K ( {h}^{- 1} x ) & ( 8 )\end{matrix}$$

In a case where a Gaussian kernel (Radius Basis Function (RBF)) is used, Expression (9) can be used.

$$\begin{matrix}[ Math . 9 ] & \\ {K}_{h} ( x ) = exp ( {h}^{- 1} {‖ x ‖}^{2} ) & ( 9 )\end{matrix}$$

E_{x to p0}[K_{t∥x0-x1∥}(x-x₀)] can be regarded as an estimated value of the probability density of the point x by kernel density estimation that focuses on the periphery of the start point x₀ and takes a larger (wider) range of interest as the value of t increases.

E_{x to p1}[K_{(1-t)∥x0-x1∥}(x-x₁)] can be regarded as an estimated value of the probability density of the point x by kernel density estimation that focuses on the periphery of the end point x₁ and takes a larger range of interest as the value of 1−t increases.

Expression (7) can be regarded as an expression for setting the average μₜ in such a way that, in a case where the point x moves from the start point x₀ to the end point x₁, the point x passes through a region having a high data density near the start point x₀ in the vicinity of t=0, and passes through a region having a high data density near the start point x₁ in the vicinity of t=1.

Alternatively, the bandwidth may be set as “t^{α}∥x₀-x₁∥” or “(1−t)^{α}∥x₀-x₁∥”. In this case, the average μₜ is represented by Expression (10).

$$\begin{matrix}[ Math . 10 ] & \\ {μ}_{t} ( {x}_{0} , {x}_{1} ) = ( 1 - t ) \times \frac{{E}_{x ∼ {p}_{0}} [ x {K}_{{t}^{α} \vert \vert {x}_{0} - {x}_{1} ‖} ( x - {x}_{0} ) ]}{{E}_{x - {p}_{0}} [ {K}_{{t}^{α} \vert \vert {x}_{0} - {x}_{1} ‖} ( x - {x}_{0} ) ]} + t \times \frac{{E}_{x ∼ {p}_{1}} [ x {K}_{{( 1 - t )}^{α} ‖ {x}_{0} - {x}_{1} ‖} ( x - {x}_{1} ) ]}{{E}_{x ∼ {p}_{1}} [ {K}_{{( 1 - t )}^{α} ‖ {x}_{0} - {x}_{1} ‖} ( x - {x}_{1} ) ]} & ( 10 )\end{matrix}$$

α is a constant of a real number set as a parameter of the bandwidth.

If 0<t<1, the larger the value of α, the narrower (smaller) the band widths “t^{α}∥x₀-x₁∥” and “(1−t)^{α}∥x₀-x₁∥”.

Therefore, it can be grasped that the larger the value of α, the easier the model generation device 100 focuses on the region close to the start point x₀ and the end point x₁, and the smaller the value of α, the easier the model generation device 100 focuses on the region away from the start point x₀ and the end point x₁. For example, the operation input unit 130 may receive a user operation for specifying the value of the parameter α, and the flow setting unit 192 may set the specified value as the parameter α of the average μₜ.

Alternatively, the expression for setting the bandwidth is not limited to the expression (7) or (10), and expressions represented by various functions f (t) in which f (0)=0 can be used.

The variance σₜ(x₀, x₁) may be set as in Expression (11).

$$\begin{matrix}[ Math . 11 ] & \\ {σ}_{t} ( {x}_{0} , {x}_{1} ) = σ ‖ {x}_{0} - {x}_{1} ‖ & ( 11 )\end{matrix}$$

σ on the right side is a constant.

Alternatively, the variance σₜ(x₀, x₁) may be set as in Expression (12).

$$\begin{matrix}[ Math . 12 ] & \\ {σ}_{t} ( {x}_{0} , {x}_{1} ) = σ ‖ {x}_{0} - {x}_{1} ‖ \sqrt{t ( 1 - t )} & ( 12 )\end{matrix}$$

The probability distribution pₜ(x|x₀, x₁) followed by the point x is expressed by the above Expression (3) using the average μₜ(x₀, x₁) and the variance σₜ(x₀, x₁).

The differential μ′ₜ(x₀, x₁) at t of the average μₜ(x₀, x₁) expressed by Expression (10) can be expressed by Expression (13).

$$\begin{matrix}[ Math . 13 ] & \\ {μ}_{t}^{′} ( {x}_{0} , {x}_{1} ) = \frac{d}{d t} [ ( 1 - t ) \times \frac{f ( t , {x}_{0} , id )}{f ( t , {x}_{0} , 1 )} + t \times \frac{f ( 1 - t , {x}_{1} , id}{f ( 1 - t , {x}_{1} , 1 )} ] = \frac{f ( 1 - t , {x}_{1} , id )}{f ( 1 - t , {x}_{1} , 1 )} - \frac{f ( t , {x}_{0} , id )}{f ( t , {x}_{0} , 1 )} + ( 1 - t ) \times ( \frac{{f}^{′} ( t , {x}_{0} , id )}{f ( t , {x}_{0} , 1 )} - \frac{f ( t , {x}_{0} , id ) {f}^{′} ( t , {x}_{0} , 1 )}{{f ( t , {x}_{0} , 1 )}^{2}} ) - t \times ( \frac{{f}^{′} ( 1 - t , {x}_{1} , id )}{f ( 1 - t , {x}_{1} , 1 )} - \frac{f ( 1 - t , {x}_{1} , id ) {f}^{′} ( 1 - t , {x}_{1} , 1 )}{{f ( 1 - t , {x}_{1} , 1 )}^{2}} ) & ( 13 )\end{matrix}$$

The function f(t, y, g) is expressed as Expression (14).

$$\begin{matrix}[ Math . 14 ] & \\ f ( t , y , g ) = {E}_{x - {p}_{0}} [ g ( x ) {K}_{t ‖ {x}_{0} - {x}_{1} ‖} ( x - y ) ] = {E}_{x ∼ {p}_{0}} [ g ( x ) exp ( \frac{{‖ x - y ‖}^{2}}{t ‖ {x}_{0} - {x}_{1} ‖} ) ] & ( 14 )\end{matrix}$$

The function “id” in Expression (13) indicates an identity mapping. id(x) =x.

The function “1” in Expression (13) indicates a constant function that maps any argument to the constant 1. 1(x)=1.

The differential f′(t, y, g) at t of the function f(t, y, g) is expressed by Expression (15).

$$\begin{matrix}[ Math . 15 ] & \\ {f}^{′} ( t , y , g ) = - {E}_{x ∼ {p}_{0}} [ g ( x ) \frac{{‖ x - y ‖}^{2}}{{t}^{2} ‖ {x}_{0} - {x}_{1} ‖} exp ( \frac{{‖ x - y ‖}^{2}}{t ‖ {x}_{0} - {x}_{1} ‖} ) ] & ( 15 )\end{matrix}$$

If σₜ=σ√(t(1−t)), σ′ₜ/σₜ is expressed by Expression (16).

$$\begin{matrix}[ Math . 16 ] & \\ \frac{{σ}_{t}^{′}}{{σ}_{t}} = \frac{1 - 2 t}{2 \sqrt{t ( 1 - t )}} σ \times \frac{1}{σ \sqrt{t ( 1 - t )}} = \frac{1 - 2 t}{2 t ( 1 - t )} & ( 16 )\end{matrix}$$

Flow uₜ(x|x₀,x₁) is expressed by Expression (17).

$$\begin{matrix}[ Math . 17 ] & \\ {u}_{t} ( x \vert {x}_{0} , {x}_{1} ) = \frac{1 - 2 t}{2 t ( 1 - t )} ( x - {μ}_{t} ) + {μ}_{t}^{′} & ( 17 )\end{matrix}$$

The optimization unit 193 can optimize the model parameter θ using Expression (17).

Even in a case of using the average μₜ(x₀, x₁) shown in Expression (10), the differential μ′ₜ(x₀, x₁) at t can be calculated, and the flow uₜ(x|x₀, x₁) can be calculated. The optimization unit 193 can optimize the model parameter θ using flow uₜ(x|x₀, x₁).

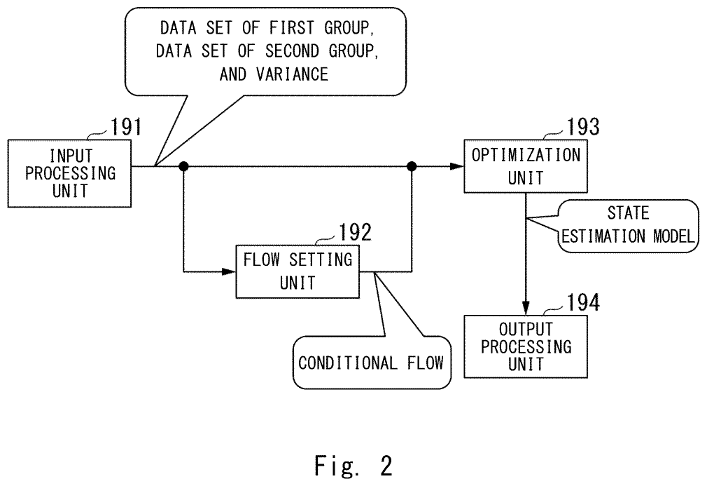

FIG. 2 is a diagram illustrating an example of data input/output in the model generation device 100.

In the example of FIG. 2, the input processing unit 191 acquires various data used for training the state estimation model, such as the data set of the first group, the data set of the second group, and the variance of the Gaussian distribution according to the point x. Then, the input processing unit 191 outputs the acquired data to the flow setting unit 192 and the optimization unit 193.

With respect to the acquisition of the variance, for example, the operation input unit 130 may receive a user operation for selecting one of a plurality of variance options such as Expression (11) and Expression (12). Then, the input processing unit 191 may output the selected variance to the flow setting unit 192 and the optimization unit 193.

Alternatively, the variance used by the model generation device 100 may be fixed to a specific one.

In this case, the storage unit 180 may store the variance. Then, the input processing unit 191 may read the variance from the storage unit 180 and output the variance to the flow setting unit 192 and the optimization unit 193. Alternatively, the flow setting unit 192 may read the variance from the storage unit 180.

The flow setting unit 192 sets the conditional flow based on the setting of the average and variance of the Gaussian distribution according to the point x. For example, the flow setting unit 192 may set the conditional flow uₜ(x|x₀, x₁) as shown in Expression (17) based on the setting of the average μₜ(x₀, x₁) shown in Expression (10) and the setting of the variance σₜ=σ√(t(1−t)).

Setting the conditional flow by the flow setting unit 192 can also be understood as defining the conditional flow.

The flow setting unit 192 outputs the set conditional flow to the optimization unit 193.

The optimization unit 193 performs optimization calculation for optimizing the parameter value of the state estimation model using the conditional flow set by the flow setting unit 192.

Specifically, for each combination of the first health condition data and the second health condition data, the optimization unit 193 optimizes the parameter value of the state estimation model so that the path of the point x indicated by the state estimation model approaches the path of the point x indicated by the conditional flow having the first health condition data value as the start point x₀ and the second health condition as the end point x₁.

The optimization unit 193 repeatedly performs optimization calculation in such a way as to optimize the parameter value of the state estimation model for all combinations of the first health condition data and the second health condition data.

In the optimization calculation, the optimization unit 193 searches for a parameter value that improves the evaluation indicated by the evaluation function shown in Expression (2) as much as possible. Specifically, the optimization unit 193 searches for a value of the parameter θ that minimizes the integral of the squared error between the output of the state estimation model vₜ(x;θ) and the output of the conditional flow uₜ(x|x₀, x₁) for the path from the start point x₀ to the ending point x₁.

However, the optimization method used by the optimization unit 193 is not limited to a specific method. For example, the optimization unit 193 may perform optimization calculation using a gradient method such as a steepest descent method, but is not limited thereto.

The optimization unit 193 outputs the trained state estimation model to the output processing unit 194. The output processing unit 194 outputs the trained state estimation model to the outside of the model generation device 100. For example, the output processing unit 194 may transmit the trained state estimation model to another device via the communication unit 110.

Alternatively, after training the state estimation model, the model generation device 100 may estimate the temporal change of the health condition data value using the trained state estimation model.

In this case, the model generation device 100 does not need to output the trained state estimation model to the outside. Therefore, the processing unit 190 may not include the output processing unit 194.

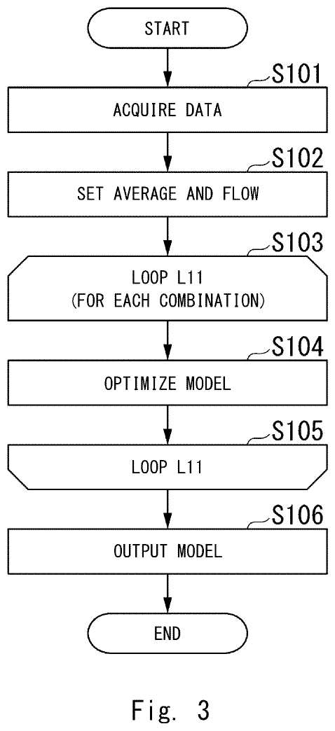

FIG. 3 is a diagram illustrating an example of a procedure of processing performed by the model generation device.

In the processing of FIG. 3, the input processing unit 191 acquires various data used for training the state estimation model (step S101).

Next, the flow setting unit 192 sets an average and a conditional flow (step S102).

In the setting of the average, the flow setting unit 192 sets a value to a parameter of an expression indicating the average, such as the parameter α of Expression (10). In a case where the expression indicating the average does not include the parameter, the flow setting unit 192 may set the expression as it is. For example, the flow setting unit 192 may read the expression indicating the average from the storage unit 180 and use the read expression as it is for the setting of the conditional flow.

In the setting of the conditional flow, the flow setting unit 192 sets an expression indicating the conditional flow as in Expression (10), for example, based on the set average and variance.

Next, the optimization unit 193 starts a loop L11 for performing processing for each combination of the first health condition data value and the second health condition data value (step S103). The first health condition data value and the second health condition data value to be processed in the loop L11 are used as a start point and an end point of the conditional flow. The first health condition data value and the second health condition data value to be processed in the loop L11 are also referred to as a fixed start point and a fixed end point.

In the processing of the loop L11, the optimization unit 193 performs optimization calculation of the parameter value of the state estimation model so that the path of the health condition data value indicated by the state estimation model approaches the path indicated by the conditional flow with the fixed start point and the fixed end point as close as possible (step S104).

For example, the optimization unit 193 samples the combination of the start point x₀ and the end point x₁ according to the probability distribution q based on Expression (2), and calculates the model vₜ(x; θ) and conditional flow uₜ(·|x₀, x₁). Then, the optimization unit 193 calculates, as an evaluation function value, an average value of squared errors in a case of moving the start point x₀ and the end point x₁, and searches for the value of the parameter θ of the model vₜ(x; θ) that will make the evaluation function value smaller.

Next, the optimization unit 193 performs termination processing of the loop L11. Specifically, the optimization unit 193 determines whether the processing of the loop L11 has been performed for all combinations (all combinations) of the first health condition data and the second health condition data.

In a case where it is determined that there is a combination for which the processing of the loop L11 has not been performed yet, the optimization unit 193 continues to perform the processing of the loop L11 on the unprocessed combination.

On the other hand, in a case where it is determined that the processing of the loop L11 has been performed on all the combinations, the optimization unit 193 ends the loop L11.

After the loop L11, the output processing unit 194 outputs the trained state estimation model (step S106).

After step S106, the model generation device 100 ends the processing of FIG. 3.

As described above, the input processing unit 191 acquires the first data set and the second data set. The first data set is a data set of first health condition data values that are health condition data values for each individual belonging to the first group. The health condition data value is a value related to the health condition. The second data set is a data set of second health condition data values that are health condition data values for each individual belonging to a second group that is a group having a temporal relationship with the first group.

The flow setting unit 192 sets the average of the Gaussian distribution for each combination of the first health condition data value and the second health condition data value, and sets the flow.

Related to the setting of the mean of the Gaussian distribution, the flow setting unit 192 sets a path of temporal change of the mean of the Gaussian distribution set as a probability distribution with which the health condition data value follows during temporal change from the first health condition data value to the second health condition data value. The flow setting unit 192 sets the path of the temporal change of the mean of the Gaussian distribution to a continuous function that connects the first health condition data value and the second health condition data value and performs weighting on the health condition data value such that the higher the data density estimated by the kernel density estimation, the larger the weight on the health condition data value.

With respect to the setting of the flow, the flow setting unit 192 sets the flow indicating the direction vector of the temporal change of the health condition data value with the first health condition data value as a start point and the second health condition data value as an end point such that the health condition data value follows the Gaussian distribution in which the average is indicated by the continuous function described above.

The optimization unit 193 performs optimization calculation of a parameter value of a model that calculates a direction vector of a temporal change of the health condition data value. The optimization unit 193 performs optimization calculation for all combinations of the first health condition data value and the second health condition data value so that the direction vector calculated using the model approaches the direction vector indicated by the flow.

According to the model generation device 100, it is not necessary to use the time series data for training the model, and in this respect, it is expected that data used for training the model can be relatively easily obtained.

According to the model generation device 100, it is possible to reflect, in training the model, the characteristic of being easily changeable through a region having a high data density as the characteristic related to the temporal change of the value related to the health condition. According to the model generation device 100, in this respect, it is expected that the model obtained by training can estimate the temporal change of the value related to human health with relatively high accuracy.

Here, as the characteristics related to the temporal change of the value related to the health condition, it is also conceivable to reflect the characteristics for each item related to the health condition, such as height, weight, and blood glucose level, in training the model. However, in this case, it is necessary to reflect the characteristics by changing the expression every time the item related to the health condition changes, which is a burden on the worker who sets the expression. For an item for which the characteristic is unknown, the characteristic related to the temporal change of the value related to the health condition cannot be reflected in training the model. In a case where the number of items related to the health condition is large, incorporating the characteristics of each of the large number of items into the expression is considered to be unrealistic because the burden on the worker who sets the expression is particularly large.

On the other hand, the model generation device 100 reflects, in training the model, a characteristic common to the item related to the health condition, that the value related to the health condition is likely to change through the region with high data density. As a result, in the model generation device 100, it is sufficient to incorporate the characteristic considered as the characteristic of the temporal change of the value related to the health condition into the expression in advance, and it is not necessary to incorporate the characteristic according to the item into the expression. According to the model generation device 100, in this respect, the burden on the operator who sets the expression can be relatively reduced.

The continuous function set as the path of the temporal change of the average of the Gaussian distribution includes a parameter for adjusting the bandwidth in the kernel density estimation.

According to the model generation device 100, it is possible to adjust which portion is emphasized as a portion having a high data density by adjusting the bandwidth by adjusting the parameter value.

For example, in a case where it is desired to cause the model generation device 100 to place importance on the vicinity of the start point x₀ and the vicinity of the end point x₁, a relatively large value may be set as the value of the parameter α of Expression (10). On the other hand, in a case where it is desired to cause the model generation device 100 to emphasize a place relatively far from the start point x₀ and the end point x₁, a relatively small value may be set as the value of the parameter α of Expression (10).

The model generation device 100 adjusts the values of the parameters of the model by machine learning.

According to the model generation device 100, a known machine learning method can be used for a part of the processing of adjusting the value of the parameter of the model. In this respect, it is expected that the model generation device 100 can be designed relatively easily.

### Second Example Embodiment

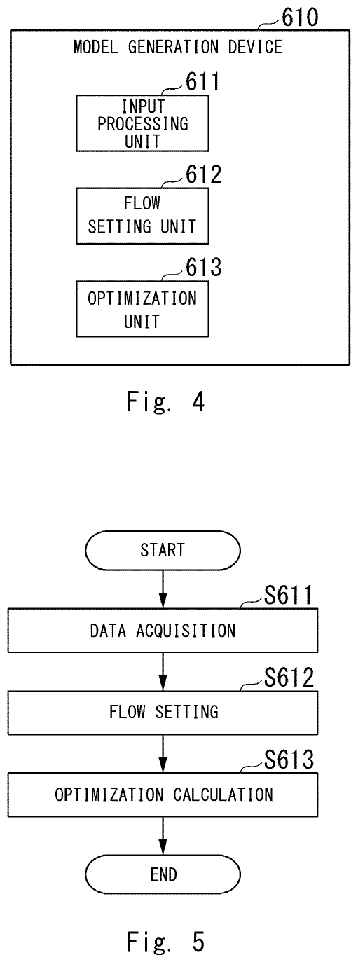

FIG. 4 is a diagram illustrating an example of a configuration of a model generation device according to at least one example embodiment.

In the configuration illustrated in FIG. 4, the model generation device 610 includes an input processing unit 611, a flow setting unit 612, and an optimization unit 613.

With such a configuration, the input processing unit 611 acquires a first data set which is a data set of first health condition data values which are health condition data values for each individual belonging to the first group among health condition data values which are values related to health conditions, and a second data set which is a data set of second health condition data values which are health condition data values for each individual belonging to the second group which is a group having a temporal relationship with the first group.

For each combination of the first health condition data value and the second health condition data value, the flow setting unit 612 sets a path of temporal change of an average of a Gaussian distribution set as a probability distribution followed by the health condition data value during temporal change from the first health condition data value to the second health condition data value, to a continuous function that connects the first health condition data value and the second health condition data value and performs weighting on the health condition data value such that the higher the data density estimated by kernel density estimation, the larger the weight on the health condition data value, and sets a flow indicating a direction vector of temporal change of the health condition data value having the first health condition data value as a start point and the second health condition data value as an end point such that the health condition data value follows the Gaussian distribution in which the average is indicated by the above-described continuous function.

The optimization unit 613 performs optimization calculation of the parameter value of the model for calculating the direction vector of the temporal change of the health condition data value such that the direction vector calculated using the model approaches the direction vector indicated by the flow for all combinations of the first health condition data value and the second health condition data value.

The input processing unit 611 is associated with an example of an input processing means. The flow setting unit 612 is associated with an example of a flow setting means. The optimization unit 613 is associated with an example of an optimization means.

According to the model generation device 610, it is not necessary to use the time series data for training the model, and in this respect, it is expected that data used for training the model can be relatively easily obtained.

According to the model generation device 610, it is possible to reflect, in training the model, the characteristic of being easily changeable through a region having a high data density as the characteristic related to the temporal change of the value related to the health condition. According to the model generation device 610, in this respect, it is expected that the model obtained by training can estimate the temporal change of the value related to human health with relatively high accuracy.

Here, as the characteristics related to the temporal change of the value related to the health condition, it is also conceivable to reflect the characteristics for each item related to the health condition, such as height, weight, and blood glucose level, in training the model. However, in this case, it is necessary to reflect the characteristics by changing the expression every time the item related to the health condition changes, which is a burden on the worker who sets the expression. For an item for which the characteristic is unknown, the characteristic related to the temporal change of the value related to the health condition cannot be reflected in training the model. In a case where the number of items related to the health condition is large, incorporating the characteristics of each of the large number of items into the expression is considered to be unrealistic because the burden on the worker who sets the expression is particularly large.

On the other hand, the model generation device 610 reflects, in training the model, a characteristic common to the item related to the health condition, that the value related to the health condition is likely to change through the region with high data density. As a result, in the model generation device 610, it is sufficient to incorporate the characteristic considered as the characteristic of the temporal change of the value related to the health condition into the expression in advance, and it is not necessary to incorporate the characteristic according to the item into the expression. According to the model generation device 610, in this respect, the burden on the operator who sets the expression can be relatively reduced.

### Third Example Embodiment

FIG. 5 is a diagram illustrating an example of a procedure of processing in the model generation method according to at least one example embodiment. The process illustrated in FIG. 5 includes acquiring data (step S611), setting a flow (step S612), and performing optimization calculation (step S613).

In acquiring data (step S611), a computer acquires a first data set which is a data set of first health condition data values which are health condition data values for each individual belonging to a first group among health condition data values which are values related to health conditions, and a second data set which is a data set of second health condition data values which are health condition data values for each individual belonging to a second group which is a group having a temporal relationship with the first group.

In setting a flow (step S612), for each combination of a first health condition data value and a second health condition data value, a computer sets a path of a temporal change of an average of a Gaussian distribution set as a probability distribution followed by the health condition data value during a temporal change from the first health condition data value to the second health condition data value to a continuous function that connects the first health condition data value and the second health condition data value and performs weighting on the health condition data value such that a weight on the health condition data value increases as a data density estimated by kernel density estimation increases, and sets a flow indicating a direction vector of the temporal change of the health condition data value with the first health condition data value as a start point and the second health condition data value as an end point such that the health condition data value follows a Gaussian distribution whose mean is indicated by the continuous function.

In performing the optimization calculation (step S613), the computer performs the optimization calculation of the parameter value of the model for calculating the direction vector of the temporal change of the health condition data value such that the direction vector calculated using the model approaches the direction vector indicated by the flow for all combinations of the first health condition data value and the second health condition data value.

According to the processing shown in FIG. 5, it is not necessary to use the time series data for training the model, and in this respect, it is expected that data used for training the model can be relatively easily obtained.

According to the processing shown in FIG. 5, it is possible to reflect, in training the model, the characteristic of being easily changeable through a region having a high data density as the characteristic related to the temporal change of the value related to the health condition. According to the processing shown in FIG. 5, in this respect, it is expected that the model obtained by training can estimate the temporal change of the value related to human health with relatively high accuracy.

Here, as the characteristics related to the temporal change of the value related to the health condition, it is also conceivable to reflect the characteristics for each item related to the health condition, such as height, weight, and blood glucose level, in training the model. However, in this case, it is necessary to reflect the characteristics by changing the expression every time the item related to the health condition changes, which is a burden on the worker who sets the expression. For an item for which the characteristic is unknown, the characteristic related to the temporal change of the value related to the health condition cannot be reflected in training the model. In a case where the number of items related to the health condition is large, incorporating the characteristics of each of the large number of items into the expression is considered to be unrealistic because the burden on the worker who sets the expression is particularly large.

On the other hand, the processing shown in FIG. 5 reflects, in training the model, a characteristic common to the item related to the health condition, that the value related to the health condition is likely to change through the region with high data density.

As a result, in the processing illustrated in FIG. 5, it is sufficient to incorporate the characteristic considered as the characteristic of the temporal change of the value related to the health condition into the expression in advance, and it is not necessary to incorporate the characteristic according to the item into the expression.

According to the processing illustrated in FIG. 5, in this respect, the burden on the operator who sets the expression can be relatively reduced.

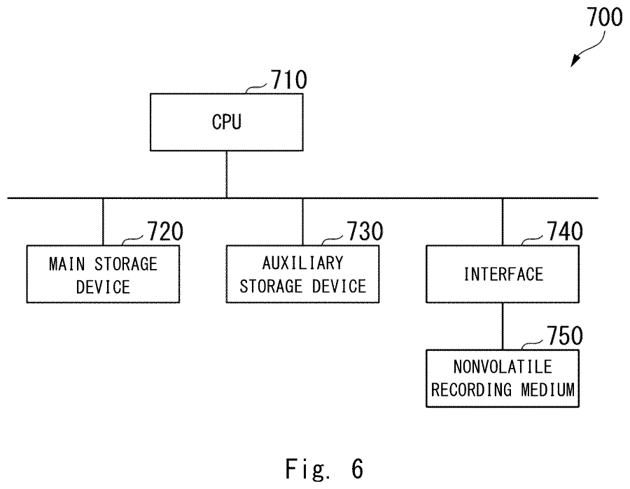

FIG. 6 is a diagram illustrating an example of a configuration of a computer according to at least one example embodiment.

In the configuration illustrated in FIG. 6, the computer 700 includes a CPU 710, a main storage device 720, an auxiliary storage device 730, an interface 740, and a nonvolatile recording medium 750.

Any one or more of the model generation device 100 and the model generation device 610 or a part thereof may be implemented in the computer 700. In this case, the operation of each processing unit described above is stored in the auxiliary storage device 730 in the form of a program. The CPU 710 reads the program from the auxiliary storage device 730, loads the program in the main storage device 720, and executes the above processing according to the program. The CPU 710 secures a storage area related to each of the above-described storage units in the main storage device 720 according to the program. Communication between each device and another device is executed by the interface 740 having a communication function and performing communication under the control of the CPU 710. The interface 740 has a port for the nonvolatile recording medium 750, and reads information from the nonvolatile recording medium 750 and writes information to the nonvolatile recording medium 750.

In a case where the model generation device 100 is implemented in the computer 700, the operations of the processing unit 190 and each unit thereof are stored in the auxiliary storage device 730 in the form of a program. The CPU 710 reads the program from the auxiliary storage device 730, loads the program in the main storage device 720, and executes the above processing according to the program.

The CPU 710 secures a storage area for the storage unit 180 in the main storage device 720 according to the program. Communication with another device by the communication unit 110 is executed by allowing the interface 740 having a communication function to be operated under the control of the CPU 710. The display of the image by the display unit 120 is executed by the interface 740 including a display device and displaying various images under the control of the CPU 710. The operation input unit 130 receives a user operation in a case where the interface 740 includes an input device and receives the user operation under the control of the CPU 710.

In a case where the model generation device 610 is implemented in the computer 700, the operations of the input processing unit 611, the flow setting unit 612, and the optimization unit 613 are stored in the auxiliary storage device 730 in the form of a program. The CPU 710 reads the program from the auxiliary storage device 730, loads the program in the main storage device 720, and executes the above processing according to the program.

The CPU 710 secures a storage area for the model generation device 610 to perform processing in the main storage device 720 according to the program. Communication between the model generation device 610 and another device is executed by the interface 740 having a communication function and operating under the control of the CPU 710. An interaction between the model generation device 610 and the user is executed in a case where the interface 740 includes an input device and an output device, information is presented to the user by the output device according to the control of the CPU 710, and a user operation is received by the input device.

Any one or more of the above-described programs may be recorded in the nonvolatile recording medium 750. In this case, the interface 740 may read the program from the nonvolatile recording medium 750. The CPU 710 may directly execute the program read by the interface 740, or may temporarily store the program in the main storage device 720 or the auxiliary storage device 730 and execute the program.

A program for executing all or part of the processing performed by the model generation device 100 and the model generation device 610 may be recorded in a computer-readable recording medium, and the processing of each unit may be performed by causing a computer system to read and execute the program recorded in the recording medium. The “computer system” herein includes hardware such as an operating system (OS) and peripheral devices.

The “computer-readable recording medium” refers to a portable medium such as a flexible disk, a magneto-optical disk, a read only memory (ROM), and a compact disc read only memory (CD-ROM), and a storage device such as a hard disk built in a computer system. The program may be for achieving a part of the functions described above, and the functions described above may be achieved in combination with a program already recorded in the computer system.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM, CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. The above-described example embodiments may be appropriately combined with other example embodiments.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example, to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

Some or all of the above-described example embodiments may be described as the following supplementary notes, but are not limited to the following supplementary notes.

### Supplementary Note 1

A model generation device including:
input processing means for acquiring a first data set that is a data set of first health condition data values that are health condition data values for each individual belonging to a first group among health condition data values that are values related to health condition, and a second data set that is a data set of second health condition data values that are health condition data values for each individual belonging to a second group that is a group having a temporal relationship with the first group;
flow setting means for setting a path of temporal change of an average of a Gaussian distribution set as a probability distribution followed by a health condition data value during temporal change from the first health condition data value to the second health condition data value for each combination of the first health condition data values and the second health condition data values, to a continuous function that connects the first health condition data value and the second health condition data value and performs weighting on the health condition data value in such a way that a weight on the health condition data value increases as a data density estimated by kernel density estimation increases, and setting a flow indicating a direction vector of temporal change of the health condition data value with the first health condition data value as a start point and the second health condition data value as an end point in such a way that the health condition data value follows a Gaussian distribution whose average is indicated by the continuous function; and
optimization means for performing optimization calculation of a parameter value of a model for calculating a direction vector of a temporal change of the health condition data value in such a way that a direction vector calculated using the model approaches a direction vector indicated by the flow for all the combinations of the first health condition data values and the second health condition data values.

### Supplementary Note 2

The model generation device according to Supplementary Note 1, in which the continuous function includes a parameter for adjusting a bandwidth in the kernel density estimation.

### Supplementary Note 3

The model generation device according to Supplementary Note 1 or 2, in which the model generation device adjusts the value of the parameter of the model by machine learning.

### Supplementary Note 4

A model generation method causing a computer to perform:
acquiring a first data set that is a data set of first health condition data values that are health condition data values for each individual belonging to a first group among health condition data values that are values related to health condition, and a second data set that is a data set of second health condition data values that are health condition data values for each individual belonging to a second group that is a group having a temporal relationship with the first group;

setting a path of temporal change of an average of a Gaussian distribution set as a probability distribution followed by a health condition data value during temporal change from the first health condition data value to the second health condition data value for each combination of the first health condition data values and the second health condition data values, to a continuous function that connects the first health condition data value and the second health condition data value and performs weighting on the health condition data value in such a way that a weight on the health condition data value increases as a data density estimated by kernel density estimation increases, and setting a flow indicating a direction vector of temporal change of the health condition data value with the first health condition data value as a start point and the second health condition data value as an end point in such a way that the health condition data value follows a Gaussian distribution whose average is indicated by the continuous function; and
performing optimization calculation of a parameter value of a model for calculating a direction vector of a temporal change of the health condition data value in such a way that a direction vector calculated using the model approaches a direction vector indicated by the flow for all the combinations of the first health condition data values and the second health condition data values.

### Supplementary Note 5

The model generation method according to Supplementary Note 2, in which the continuous function includes a parameter for adjusting a bandwidth in the kernel density estimation.

### Supplementary Note 6

The model generation method according to Supplementary Note 4 or 5, in which the computer adjusts the value of the parameter of the model by machine learning.

### Supplementary Note 7

A program causing a computer to execute:
acquiring a first data set that is a data set of first health condition data values that are health condition data values for each individual belonging to a first group among health condition data values that are values related to health condition, and a second data set that is a data set of second health condition data values that are health condition data values for each individual belonging to a second group that is a group having a temporal relationship with the first group;
setting a path of temporal change of an average of a Gaussian distribution set as a probability distribution followed by a health condition data value during temporal change from the first health condition data value to the second health condition data value for each combination of the first health condition data values and the second health condition data values, to a continuous function that connects the first health condition data value and the second health condition data value and performs weighting on the health condition data value in such a way that a weight on the health condition data value increases as a data density estimated by kernel density estimation increases, and setting a flow indicating a direction vector of temporal change of the health condition data value with the first health condition data value as a start point and the second health condition data value as an end point in such a way that the health condition data value follows a Gaussian distribution whose average is indicated by the continuous function; and
performing optimization calculation of a parameter value of a model for calculating a direction vector of a temporal change of the health condition data value in such a way that a direction vector calculated using the model approaches a direction vector indicated by the flow for all the combinations of the first health condition data values and the second health condition data values.

### Supplementary Note 8

The program according to Supplementary Note 5, in which the continuous function includes a parameter for adjusting a bandwidth in the kernel density estimation.

### Supplementary Note 9

The program according to Supplementary Note 7 or 8, further causing the computer to adjust the value of the parameter of the model by machine learning.

1. A model generation device comprising:
at least one memory storing instructions, and
at least one processor configured to execute the instructions to;
acquire a first data set that is a data set of first health condition data values that are health condition data values for each individual belonging to a first group among health condition data values that are values related to health condition, and a second data set that is a data set of second health condition data values that are health condition data values for each individual belonging to a second group that is a group having a temporal relationship with the first group;
set a path of temporal change of an average of a Gaussian distribution set as a probability distribution followed by a health condition data value during temporal change from the first health condition data value to the second health condition data value for each combination of the first health condition data values and the second health condition data values, to a continuous function that connects the first health condition data value and the second health condition data value and performs weighting on the health condition data value in such a way that a weight on the health condition data value increases as a data density estimated by kernel density estimation increases, and set a flow indicating a direction vector of temporal change of the health condition data value with the first health condition data value as a start point and the second health condition data value as an end point in such a way that the health condition data value follows a Gaussian distribution whose average is indicated by the continuous function; and
perform optimization calculation of a parameter value of a model for calculating a direction vector of a temporal change of the health condition data value in such a way that a direction vector calculated using the model approaches a direction vector indicated by the flow for all the combinations of the first health condition data values and the second health condition data values.

2. The model generation device according to claim 1, wherein the continuous function includes a parameter for adjusting a bandwidth in the kernel density estimation.

3. The model generation device according to claim 1, wherein the model generation device adjusts the value of the parameter of the model by machine learning.

4. A model generation method causing a computer to perform:
acquiring a first data set that is a data set of first health condition data values that are health condition data values for each individual belonging to a first group among health condition data values that are values related to health condition, and a second data set that is a data set of second health condition data values that are health condition data values for each individual belonging to a second group that is a group having a temporal relationship with the first group;
setting a path of temporal change of an average of a Gaussian distribution set as a probability distribution followed by a health condition data value during temporal change from the first health condition data value to the second health condition data value for each combination of the first health condition data values and the second health condition data values, to a continuous function that connects the first health condition data value and the second health condition data value and performs weighting on the health condition data value in such a way that a weight on the health condition data value increases as a data density estimated by kernel density estimation increases, and setting a flow indicating a direction vector of temporal change of the health condition data value with the first health condition data value as a start point and the second health condition data value as an end point in such a way that the health condition data value follows a Gaussian distribution whose average is indicated by the continuous function; and
performing optimization calculation of a parameter value of a model for calculating a direction vector of a temporal change of the health condition data value in such a way that a direction vector calculated using the model approaches a direction vector indicated by the flow for all the combinations of the first health condition data values and the second health condition data values.

5. The model generation method according to claim 4, wherein the continuous function includes a parameter for adjusting a bandwidth in the kernel density estimation.

6. The model generation method according to claim 4, wherein the computer adjusts the value of the parameter of the model by machine learning.

7. A non-transitory computer-readable medium storing a program that causes a computer to execute:
acquiring a first data set that is a data set of first health condition data values that are health condition data values for each individual belonging to a first group among health condition data values that are values related to health condition, and a second data set that is a data set of second health condition data values that are health condition data values for each individual belonging to a second group that is a group having a temporal relationship with the first group;
setting a path of temporal change of an average of a Gaussian distribution set as a probability distribution followed by a health condition data value during temporal change from the first health condition data value to the second health condition data value for each combination of the first health condition data values and the second health condition data values, to a continuous function that connects the first health condition data value and the second health condition data value and performs weighting on the health condition data value in such a way that a weight on the health condition data value increases as a data density estimated by kernel density estimation increases, and setting a flow indicating a direction vector of temporal change of the health condition data value with the first health condition data value as a start point and the second health condition data value as an end point in such a way that the health condition data value follows a Gaussian distribution whose average is indicated by the continuous function; and
performing optimization calculation of a parameter value of a model for calculating a direction vector of a temporal change of the health condition data value in such a way that a direction vector calculated using the model approaches a direction vector indicated by the flow for all the combinations of the first health condition data values and the second health condition data values.

8. The non-transitory computer-readable medium according to claim 7, wherein the continuous function includes a parameter for adjusting a bandwidth in the kernel density estimation.

9. The non-transitory computer-readable medium according to claim 7, wherein the program causes the computer to adjust the value of the parameter of the model by machine learning.
